(19) United States
(12) Patent Application Publication
(10) Pub. No.: US 2003/0211159 A1
(43) Pub. Date: Nov. 13, 2003

(54) Method for reduction of residual organic solvent in carbomer

(75) Inventors: David Joseph Bochniak, Southbury, CT (US); Cecile N. Forness, Danbury, CT (US); Stephen Horhota, Brookfield, CT (US); Said Saim, New Milford, CT (US)

Correspondence Address: BOEHRINGER INGELHEIM CORPORATION, 900 RIDGEBURY ROAD, P O BOX 368, RIDGEFIELD, CT, 06877, US

(73) Assignee: Boehringer Ingelheim Pharmaceuticals, Inc., Ridgefield, CT

(21) Appl. No.: 10/419,436

(22) Filed: Apr. 21, 2003

Related U.S. Application Data
(60) Provisional application No. 60/374,919, filed on Apr. 23, 2002.

(51) Int. Cl. A61K 38/28; A61K 38/00; A61K 31/56; A61K 9/14; C08J 3/00

(52) U.S. Cl. 424/486; 528/501; 514/291; 514/2; 514/171; 514/3

(57) ABSTRACT

Method for reducing the level of residual organic solvent in a carbomer comprising exposing a carbomer containing residual organic solvent to a gaseous fluid in which said residual organic solvent is substantially soluble and under conditions sufficient to extract at least some of the residual organic solvent from the carbomer; carbomers treated by this method and pharmaceutical suspensions containing the treated carbomer and a therapeutically active agent. This method is effective for the reduction of residual organic solvent in carbomer down to the ppm level, e.g., less than 30 ppm, preferably less than 10 ppm, more preferably less than 2 ppm of residual organic solvent.

This application claims the benefit of U.S. Provisional Application No. 60/374,919, filed on Apr. 23, 2002, which application is herein incorporated by reference in its entirety.

### FIELD OF INVENTION

This invention relates to methods for the reduction of residual organic solvent in carbomers.

### BACKGROUND OF THE INVENTION

Polymers are widely used in the chemical industry. One family of polymers are high molecular weight, cross-linked, acrylic acid-based compounds which in aqueous solutions form hydrogels. The generic (non-proprietary) name “carbomer” has been adopted by various regulatory entities for a class of homopolymers. For example, the United States Pharmacopoeia (USP-NF), the European Pharmacopoeia (EP), British Pharmacopoeia, United States Adopted Names Council (USAN), the International Nomenclature for Cosmetic Ingredients (INCI), the Japanese Pharmaceutical Excipients list, and the Italian Pharmacopoeia all have adopted the name “carbomer”. Carbomer homopolymers are polymers of acrylic acid cross-linked with a variety of compounds including, but not limited to, allyl sucrose and allylpentaerythritrol (the so-called Carbopol® polymers), divinyl glycol, or copolymers of acrylic acid with various amounts of long-chain alkyl acrylate co-monomers cross-linked with allylpentaerythritrol, for example. Practitioners skilled in the art recognize that carbomers commonly have notations in the name to indicate various chemico-physical properties. Accordingly, “Carbomer 934” is distinguished from “Carbomer 1342” or “Carbomer 934P”.

Residual organic solvents are organic solvents that are not completely removed from chemical compounds during their manufacture. Practitioners in the art readily appreciate that such manufactured chemical compounds that may contain residual organic solvents as a result of their manufacturing process include, for example, drug substances or drug excipients. Examples of residual organic solvents that might be present include, for example, benzene, phenol(s), toluene, ethyl acetate, methanol, ethanol, isopropanol, hexane, acetone, chloroform, 1,4-dioxane, dimethyl sulfoxide, methylene chloride, trichloroethylene, 1,2-dichloroethane, carbon tetrachloride, and 1,1-dichloroethene. Appropriate selection of the solvent for the synthesis of excipient or drug substance may enhance the yield, or determine the characteristics such as crystal form, solubility and purity. Therefore, the selection of solvent may sometimes be a critical choice in the synthetic process. In the pharmaceutical industry, however, since there is no therapeutic benefit from residual organic solvents and their presence at high levels could be harmful, all residual organic solvents should be removed to the lowest extent practicable. The U.S. Food and Drug Administration has identified benzene as a solvent with unacceptable toxicity. Based upon the International Conference on Harmonization of Technical Requirements for Registration of Pharmaceuticals (ICH) Impurities Guideline for Residual Solvents, the EP concentration limit for benzene is no more than 2 ppm in a pharmaceutical excipient. Additionally, the FDA has promulgated a guidance concentration limit for benzene of 2 ppm in a drug product (see FR Doc. 97-33639).

Carbomer 934P, e.g., Carbopol® 934P (BF Goodrich/Noveon), is a high molecular weight polyacrylic anionic polymer cross linked with allyl sucrose and is widely used as a thickening agent in pharmaceutical preparations. For example, Carbopol® 934P is presently used as the suspending/thickening agent in Viramune® (nevirapine) oral suspension useful for anti-HIV therapy. However, benzene is used as a solvent in the manufacture of Carbopol® 934P. As a result, commercial supplies of Carbopol® 934P have benzene concentration levels that exceed the allowable limit specified in the EP. Therefore, either the Carbopol® 934P must be replaced with an alternative carbomer having an acceptable level of residual organic solvent or a feasible method must be developed to reduce the level of benzene in Carbopol® 934P. Both these options were investigated during the development of the present invention.

Carbomers have been described and used since 1955 (Swafford, W. B, Nobles, L. W., “Some Pharmaceutical Uses of Carbopol 934,” Journal of the American Pharmaceutical Association, 16(3), March 1955). As is well established in the art, a carbomer can be used by first dispersing it in water. Subsequent addition of a base such as sodium hydroxide causes the polymer to uncoil and form a viscous gel matrix. This viscous gel matrix serves as a thickening agent for pharmaceutical suspensions. For pharmaceutical suspensions, gel viscosity is an essential characteristic in pharmaceutical manufacturing, and gel viscosity must be controlled and have little batch-to-batch variability in order to achieve the desired therapeutic benefit of the drug substance uniformly dispersed and suspended in the gel matrix. The gel viscosity depends on three factors: intrinsic carbomer viscosity, carbomer concentration, and neutralization pH (extent of ionization) (Noveon, Bulletin 11 Thickening Properties, January, 2002, FIGS. 11.1.2 and 11.2.2). These factors are the key functionality components of the carbomer. For the alternate carbomers evaluated as possible replacements to Carbopol® 934P in the Viramune® oral suspension product, for example, the intrinsic carbomer viscosity range or the effect of ions/pH on viscosity was not sufficiently similar to that of Carbopol® 934P to assure that the desired viscosity would be consistently achieved in the drug suspension. Therefore, replacement of a particular carbomer with an alternate carbomer was not a straightforward solution to this problem. The present invention overcomes the need to exhaustively search for acceptable alternatives, however, since it allows for the reduction of residual organic solvents in a selected carbomer without adversely affecting carbomer properties and functionality.

In addition to the viscosity issues regarding carbomer functionality, the dispersion of the carbomer is also critical to achieving a uniform product. Carbomer is commercially supplied as a fine particulate powder and as such, it tends to be difficult to disperse. Ideally, discrete particles of carbomer should be wetted in the solvent media. Unlike other powders in which lumped masses can eventually be reduced, if carbomer agglomerates, then the surface will solvate forming an external gel layer which prevents wetting of the interior powder and dispersion. Consequently, a uniform dispersion is not achieved, and the agglomeration of un-neutralized carbomer in the gel matrix could result in a non-uniform suspension of lower viscosity. Until the present invention, and as explained more fully below, traditional methods used to reduce residual solvents to very low levels would change the physical structure of the carbomer thereby causing difficulty with dispersion and therefore difficulty achieving the necessary viscosity. Using the process of the present invention, however, the integrity of the physical structure of the carbomer is sufficiently maintained.

Traditional methods of solvent removal, such as drying, are not effective in eliminating benzene from carbomer while maintaining the integrity of the material. Removal of an organic solvent such as benzene from a particle by conventional means such as by drying depends upon its rate of diffusion from within the particle and its vapor pressure. Solute diffusion in a solid, nonporous particle is generally slow and high temperatures are often required to enhance the diffusion rates. Unfortunately, high temperatures can result in degradation of polymer particles (Noveon Bulletin 5 Polymer Handling and Storage, January 2002, p. 1). The manufacturer of Carbopol® 934P discloses that when the drying process is modified to reduce residual benzene below the EP limit, the modified drying process causes sintering of the carbomer thereby rendering the carbomer difficult to rehydrate. It was therefore doubtful that any straightforward method to reduce residual benzene such as by evaporation at atmospheric pressure or under vacuum would be successful at removing benzene from within the carbomer matrix while maintaining the functionality of the carbomer. Therefore, there existed a need to develop methods that could reduce the level of residual solvent in carbomers while sufficiently maintaining the integrity of their chemico-physical properties.

A known method for reducing solvent in polymers is Supercritical Fluid Extraction (SFE). For example, Hoffman et al. (U.S. Pat. No. 5,607,518) disclose a process for removing residual solvents from polymeric materials such as contact lenses. Duda et al. (U.S. Pat. No. 5,917,011) disclose a process whereby fluid pressure is cycled to remove impurities from polymeric substrates. Horhota et al. disclose methods for removing soluble material from confined spaces within substrates such as containers, capsules and porous powders (U.S. Pat. Nos. 6,294,194 B1 and 6,228,394 B1). Supercritical fluids (SCFs) have been reported to be useful in other extraction applications including re-dissolution of adsorbed material (U.S. Pat. No. 4,061,566), the formation of porous polymers, removal of residual solvents from articles formed by compression such as tablets (U.S. Pat. No. 5,287,632), monomer purification and fractionation of various polymers.

A substantial discussion of the many uses to which SCFs have been employed is set forth in the text Supercritical Fluid Extraction by Mark McHugh and Val Krukonis (Butterworth-Heinmann 1994). The extraction solvent used in the SFE process is a gaseous fluid, such as carbon dioxide (CO₂), sulfur dioxide, or nitrous oxide, generally at a temperature and/or pressure above its critical temperature and pressure. SFE takes advantage of gas-like diffusivity and liquid-like solvent power of supercritical fluids to dissolve and extract solutes from confined spaces. Although polymers can be solid, non-porous material, dissolution of a supercritical fluid in a polymer matrix can serve to plasticize the polymer and increase the mobility of solvent molecules thereby enhancing the removal and the rate of extraction of the residual solvent.

At the time of the present invention, however, SFE had not been used for the reduction of residual organic solvent in carbomers. Since using gases such as carbon dioxide, hydrogen or sulfur dioxide under pressure is a common technique for inducing chemical reactions, it would have been expected that a carbomer undergoing SCF extraction with a pressurized gaseous fluid would undergo chemical changes resulting in an altered chemical and physical structure. For example, it has been reported (See Ikushima, Y., Advances in Colloid & Interface Science, 1997, 71-72:259-280) that a biopolymer (enzyme) undergoes drastic conformational changes with exposure to supercritical CO₂ in the near-critical region. The induction of a similar conformational change in carbomers during exposure to supercritical CO₂ could interfere with the uncoiling of the polymer and gel development upon hydration and neutralization of the carbomer in the usual manner. Therefore, at the time of the present invention it would not have been expected that SFE would be a viable alternative to reduce the level of organic solvents in carbomers.

### SUMMARY OF THE INVENTION

With the present invention, it was unexpectedly discovered that SFE processing of a carbomer could be used to reduce the level of residual organic solvent in a carbomer while at the same time sufficiently maintaining the carbomer's physical structure and functionality.

Therefore, in one embodiment the present invention is directed to a method for reducing the level of residual organic solvent in a carbomer comprising exposing a carbomer containing residual organic solvent to a gaseous fluid in which said residual organic solvent is substantially soluble and under conditions sufficient to extract at least some of the residual organic solvent from the carbomer.

As described more fully hereinafter, the method of the present invention has broad applicability and can be used to extract a wide variety of residual organic solvents from carbomers under a variety of SFE processing conditions, i.e., using various types of gaseous fluids and processing conditions appropriate for the residual organic solvent(s) to be extracted from the carbomer. In addition, the processing conditions can include extraction under a constant pressure of gaseous fluid or under pressure modulation in which the pressure level of the gaseous fluid is made to modulate between two or more pressure levels during the extraction.

As also described hereinafter, the method of the present invention can be used to reduce the residual organic solvent to a variety of levels depending upon the processing conditions. In particular, the residual organic solvent(s) can be reduced to levels below the allowable limits set by the various regulatory agencies. For example, benzene can be reduced to a level below the 2 ppm level set by the EP.

In yet additional embodiments, the present invention is directed to a carbomer that has been treated by the above method, and a suspension comprising the treated carbomer and a therapeutically active agent.

### BRIEF DESCRIPTION OF THE DRAWING

FIG. 1 is a schematic diagram of an experimental supercritical fluid extraction apparatus.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below:

By the term “about” with respect to a recited value is meant ±20% of the recited value, preferably ±10%, more preferably ±5%, even more preferably ±1%. When the term “about” is used in relation to a range of values, the term “about” is intended to qualify each recited end-point of the range. For example, the phrase “about 0.8 to 1.4 T_{c}” is equivalent to “about 0.8 to about 1.4 T_{c}”.

By “residual organic solvent” is meant an organic solvent that is not completely removed from chemical compounds during their manufacture. Examples of residual organic solvents that might be present include, for example, benzene, phenol(s), toluene, ethyl acetate, methanol, ethanol, isopropanol, hexane, acetone, chloroform, 1,4-dioxane, dimethyl sulfoxide, methylene chloride, trichloroethylene, 1,2-dichloroethane, carbon tetrachloride, and 1,1-dichloroethene, as well as other organic solvents typically used in the manufacture of therapeutically active agents or pharmaceutical excipients.

By “gaseous fluid”, or “supercritical fluid” is meant (1) a fluid or mixture of fluids that is gaseous under atmospheric conditions and that has a moderate critical temperature (i.e., ≦200° C.), or (2) a fluid that has previously found use as a supercritical fluid. Examples of specific gaseous fluids useful in the present method are described below. Unless explicitly stated, the temperature and pressure of the gaseous or supercritical fluid can be anywhere in the near-critical to supercritical region, e.g., in the range of about 0.8-1.4 T_{c} and about 0.5-100 P_{c} where T_{c} and P_{c} are, respectively, the critical temperature in K and the critical pressure of the fluid.

By the term “substantially soluble”, e.g., with respect to the solubility of the residual organic solvent in the gaseous fluid, is meant that under selected processing conditions the residual organic solvent can be completely solubilized by the gaseous fluid with the exception of a small quantity of residual organic solvent contamination that may be present on the carbomer particles. Quantitatively, it is preferable that at least about 95%, more preferably at least about 99%, of the residual organic solvent is solubilized in the gaseous fluid.

The method of the present invention is useful for reducing the level of residual organic solvent that may be present in a wide variety of carbomers. Examples of carbomers that may be treated by the present inventive method include, for example, carbomer 934, carbomer 934P, carbomer 940, carbomer 941, carbomer 1342, polycarbophil, and calcium polycarbophil. Commercially available carbomers include the various Carbopol® polymers from Noveon, Inc., such as Carbopol® 934P.

Examples of residual organic solvents that may be present in a carbomer and that can be extracted by the present inventive method include, for example, benzene, phenol(s), toluene, ethyl acetate, methanol, ethanol, isopropanol, hexane, acetone, chloroform, 1,4-dioxane, dimethyl sulfoxide, methylene chloride, trichloroethylene, 1,2-dichloroethane, carbon tetrachloride, and 1,1-dichloroethene.

The gaseous fluid employed in the inventive method includes, for example, any gaseous fluid that is commonly employed in conventional supercritical fluid processes such as SFE. Preferably, the gaseous fluid used has a critical temperature less than about 200° C. and a critical pressure of less than about 10,000 psi. Any suitable gaseous fluid may be used in the described processes, including, but not limited to carbon dioxide, nitrous oxide, sulfur hexafluoride, trifluoromethane, tetrafluoromethane, ethane, ethylene, propane, propanol, isopropanol, propylene, butane, butanol, isobutane, isobutene, hexane, cyclohexane, benzene, toluene, o-xylene, ammonia, water, and mixtures thereof. A preferred gaseous fluid is carbon dioxide.

Organic solvent modifiers may also be added to any of the gaseous fluids to modify their solvent properties, including, but not limited to, ethanol, methanol, acetone, propanol, isopropanol, dichloromethane, ethyl acetate, dimethyl sulfoxide, and mixtures thereof. Organic solvent modifiers are used preferably at relatively low concentrations (0 - 20%). Similarly, light gases such as N₂, O₂, He, air, H₂, CH₄ and mixtures thereof may also be added in various proportions to the gaseous fluids to alter its extraction or transport properties. Methods for determining these parameters are known to persons of ordinary skill in the art.

The method of the present invention can be conducted at near-critical and supercritical conditions where the temperature is in the range of about 0.8-1.4 T_{c}, where T_{c} is the critical temperature in K of the gaseous fluid, and the pressure is in the range of about 0.5-100 P_{c}, where P_{c} is the critical pressure of the gaseous fluid . Hence, the gaseous fluid in either its subcritical or supercritical state may be used. Extraction may be conducted in a direct manner; by mixing the vessel content while contacting the material to be extracted with the gaseous fluid; by fluidizing the material to be extracted with the gaseous fluid; or by a pressure modulation SFE method as described in more detail below. Preferably, the extraction is conducted within a temperature range of about 1.0-1.2 T_{c}, and a pressure in the range of about 1-9 P_{c}. In the case of extraction with carbon dioxide, a temperature of about 31-80° C. and a pressure of about 1,070-10,000 psig are preferred. The method of the invention may be practiced either isothermally or not.

The method of the present invention can be conducted at either a constant pressure (i.e., the pressure of the gaseous fluid is kept constant during the extraction process) or under pressure modulation (i.e., the pressure of the gaseous fluid is repeatedly modulated between two or more pressure levels during the extraction of the organic solvent). Such SFE methods that may be used in the present invention include the SFE methods as described more fully in Horhota et al, U.S. Pat. Nos. 6,228,394 B1 and 6,294,194 B1, both of which are herein incorporated by reference in their entirety. If a pressure modulation technique is used, it is preferred that the relative difference between the uppermost and lowermost levels of density of said gaseous fluid at said pressure levels is not more than about 30%, more preferably not more than about 5%. The method of control of pressure can be either manual or automatic. On/off automatic pressure control is preferred. The pressure profile may resemble a horizontal line, sync wave, a square wave, or other profile.

The vessel used to perform the extraction can vary in size and shape and may also include a mixing device. Mixing may be employed throughout the SFE process or only during specific phases of the process. The mixer can be operated continuously or intermittently and the mixing speed may also be fixed or varied.

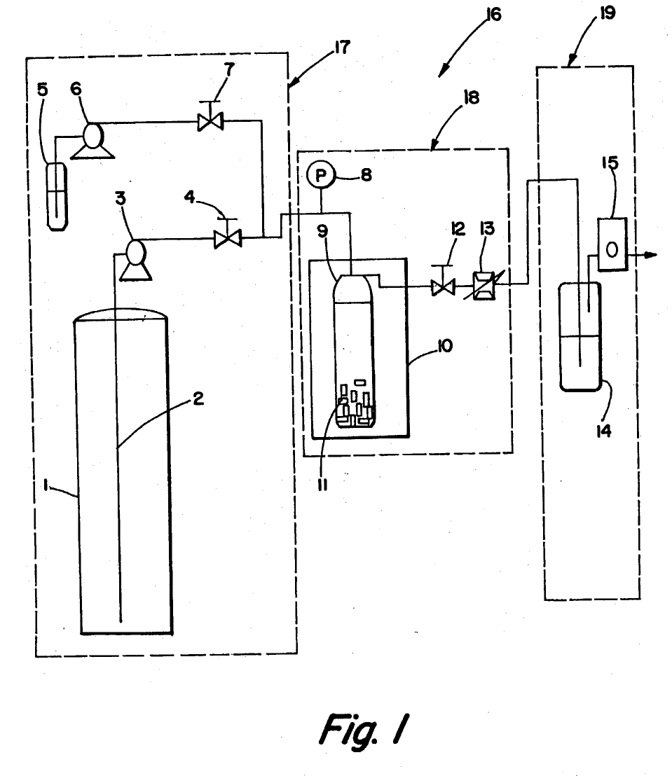

Now turning to the illustration, there is shown in FIG. 1 a conventional SFE unit generally designated by 16. Unit 16 may be characterized as comprising three main sections: feed section 17, extraction section 18, and extract recovery and flow measurement section 19. In a typical operation, a known amount of material 11 (e.g., carbomer) to be subjected to the extraction process is loaded into extraction vessel 9. Extraction vessel 9 is then placed in an isothermal oven 10. Liquid gaseous fluid (e.g., liquid CO₂) from cylinder 1 is subsequently pumped through siphon tube 2 from gaseous fluid cylinder 1 at a constant rate through pump 3 (which is preferably an air-driven pump or a metering pump fitted with a cooled head), and shut-off valve 4. Effluent shutoff valve 12 is initially kept closed until pressure in extraction vessel 9 reaches the desired extraction pressure. Additive may be added to the gaseous fluid entering extraction vessel 9 from additive container 5, by way of pump 6 and valve 7. When the desired pressure is reached, effluent shutoff valve 12 is opened and flow through, heated metering valve 13 and flow meter or totalizer 15 is established. Pressure is then either maintained constant at that pressure level or made to oscillate between two pressure levels continuously with a relatively constant frequency of pressure modulation. Pressure in extraction vessel 9 may be monitored either electronically or using pressure gauge 8.

In application of the present invention, pressure/density may be modulated between levels by merely changing inlet air pressure to the pump while keeping effluent gaseous fluid flow rate approximately constant. Pressure modulation may be effected using other ways, including (1) repeatedly reducing pump flow rate while maintaining effluent flow rate relatively constant until pressure reaches the lower level and then increasing pump flow rate to effect a pressure buildup; and (2) repeatedly closing valve 12 to allow for pressure buildup and then opening it to allow for an effluent flow rate that is higher than pump flow rate.

Following expansion through the metering valve 13, gaseous fluid is vented out near atmospheric pressure. The extract may be recovered in vessel 14, for example, by use of a cold trap consisting of a vial immersed in ice or dry ice. At the end of the extraction period, pressure is typically allowed to slowly decrease to atmospheric level. The residue in the vessel is then weighed and prepared for analysis if applicable. The material 11 that has been subjected to extraction (e.g., the treated carbomer) is then recovered from the extraction vessel 9. As would be recognized by one of ordinary skill in the art, variations in the described experimental procedure are possible, including the possibility of holding the pressure constant for some time prior to reducing pressure, i.e. using a hold time period. The gaseous fluid may be vented to higher pressure than atmospheric level and may alternatively be recycled into the process.

In some instances it has been found that carbomer treated by the method of the present invention will have a tendency to agglomerate to form an aggregate or cake rather than the desired powdered carbomer product. In this situation, it may be necessary or desirable to add another processing step(s) (e.g., grinding or milling) to break up any clumps or cakes prior to using the treated carbomer in a suspension. The present invention contemplates and includes the possibility of such further optional processing step(s) as may be necessary or desirable in a particular process.

Several SFE units are commercially available from companies such as ISCO, Inc. (Lincoln, Nebr.) which markets analytical scale SFE units and Applied Separations (Allentown, Pa.) which markets both small scale as well as semi-pilot scale SFE units. Any of these kinds of units could be used for this process. In the experimental examples set forth below, an Applied Separations lab-scale unit was used.

Any person skilled in the use of SCFs and SFE will realize that variations in this experimental procedure are possible. Depending upon the residual organic solvent that is desired to be removed from the carbomer to be treated, and following the procedures as described herein, one skilled in SFE could readily determine the gaseous fluid and experimental conditions that would be sufficient to extract at least some of the residual organic solvent from the carbomer. The optimum conditions for a particular extraction procedure to reduce a specific residual organic solvent to a desired level can be readily determined by one skilled in SFE techniques. In one embodiment, carbon dioxide has been found to be a preferred gaseous fluid for the extraction of benzene from carbomer 934P.

The method of the present invention can be used to reduce the level of residual organic solvent in a carbomer to the ppm level, e.g., less than about 30 ppm, preferably less than about 10 ppm, more preferably less than about 2 ppm.

In one preferred embodiment of the present method, carbon dioxide is used as the gaseous fluid to reduce the level of benzene in a carbomer, e.g., carbomer 934P. This preferred method can also be performed at either a constant pressure or using the pressure modulation, and the level of residual benzene in the carbomer can be reduced to the ppm level, e.g., less than about 30 ppm, preferably less than about 10 ppm, more preferably less than about 2 ppm of benzene.

The present invention is also directed to a carbomer that has been treated by any of the above described methods of the present invention, and to a suspension comprising the treated carbomer and a therapeutically active agent.

In preferred embodiments, the therapeutically active agent of the suspension can be selected from known therapeutically active agents, such as meloxicam, ipratropium bromide, tiotropium bromide, oxytropium bromide, albuterol, albuterol sulfate, clenbuterol, fenoterol, beclomethasone diproprionate, insulin, amino acids, analgesics, anti-cancer agents, antimicrobial agents, antiviral agents such as nevirapine (Viramune®) antifungals, antibiotics, nucleotides, amino acids, peptides, proteins, immune suppressants, thrombolytics, anticoagulants, central nervous system stimulants, decongestants, diuretic vasodilators, antipsychotics, neurotransmitters, sedatives, hormones, anesthetics, anti-inflammatories, antioxidants, antihistamines, vitamins, minerals and other therapeutically active agents known to the art that would be administrable by suspension. The preferred suspension comprises the treated carbomer, e.g., treated carbomer 934P, and nevirapine. Of course, conventional pharmaceutically acceptable carriers, excipients and/or other additives may be included in the suspension to prepare optimized formulations. The selection of appropriate additional carriers, excipients and/or other additives, and amounts thereof, for any particular suspension could be readily determined by a person skilled in pharmaceutical formulation techniques.

In order that this invention be more fully understood, the following examples of are set forth. These examples are for the purpose of illustrating embodiments of this invention, and are not to be construed as limiting the scope of the invention in any way.

### EXAMPLE

SFE Extraction of Benzene from Carbomer 934P

Laboratory scale SFE experiments using CO₂ were performed focusing on the process parameters of time, temperature, and pressure in order to determine a process method for reducing the benzene level in the carbomer below 2 ppm while still maintaining functionality. Pressure modulation experiments were also performed to evaluate the effectiveness of that method of processing. The residual benzene level in SFE-treated samples of Carbomer 934P was measured by HPLC assay per USP 24 method for direct injection. SFE treated carbomer functionality was checked by preparing placebo suspensions and visually monitoring the dispersion of the carbomer in water, measurement of gel pH, and suspension pH and viscosity.

The Carbopol® 934P used for the SFE evaluation, Lot BB16556, had an initial benzene concentration of 67 ppm according to the vendor Certificate of Analysis. The results of lab-scale SFE feasibility experiments are summarized in TABLE 1 below. The visual observations of SFE treated Carbopol® 934P are included to provide an indication of the material consistency after processing. As TABLE 1 indicates, all of the trials were successful at reducing the residual benzene concentration in the carbomer and placebo suspension of acceptable viscosity could be produced with all of the samples. The results of trial 4340p050 show that the residual benzene concentration was reduced below the target level of 2 ppm, to 1.3 ppm, while maintaining its functionality.

TABLE 1
Summary of Supercritical Fluid Extraction of Benzene from Carbomer 934P

| | | | | Placebo | |
|---|---|---|---|---|---|
| SFE | | SFE Treated | Benzene | Suspension | |
| Trial | | Carbomer 934P | Assay | | Viscosity |
| Number | SFE Parameters | Observations | (ppm) | pH ‡ | cp*† |
| 4340p032 | 35° C., 170 bar, 2 hours | Powder flow improved | 24.6 | 5.98 | 677 |
| 4340p035 | 55° C., 170 bar, 2 hours | Powder flow improved; | 18.1 | 5.89 | 816 |
| | | powder adhered to walls | | | |
| 4340p039 | 35° C., pressure | Powder adhered to walls | 52.0 | 5.90 | 799 |
| | modulation from 200 to | | | | |
| | 140 bar (210 swings), 2 | | | | |
| | hours | | | | |
| 4340p045 | 85° C., pressure | Majority of carbomer | 9.2 | 6.00 | 818 |
| | modulation from 300 to | formed a cylindrical | | | |
| | 600 bar (70 swings), 133 | aggregate; | | | |
| | min. | Carbomer was ground | | | |
| | | using mortar and pestle | | | |
| | | prior to suspension | | | |
| | | preparation | | | |
| 4340p050 | 55° C., 170 bar, 4 hours | Majority of carbomer | 1.3 | 6.03 | 838 |
| | | electrostatic powder, small | | | |
| | | cake present | | | |

‡Viramune ® specification for pH is 5.5-6.0
*Brookfield Spindle 3 @ 100 rpm,
†Viramune ® specification for viscosity in Europe is not less than 500 cp.

While the invention has been described with respect to preferred embodiments, those skilled in the art will readily appreciate that various changes and/or modifications can be made to the invention without departing from the spirit or scope of the invention.

What is claimed is:

1. A method for reducing the level of residual organic solvent in a carbomer comprising exposing a carbomer containing residual organic solvent to a gaseous fluid in which said residual organic solvent is substantially soluble and under conditions sufficient to extract at least some of the residual organic solvent from the carbomer.

2. A method according to claim 1, wherein the carbomer is selected from carbomer 934, carbomer 934P, carbomer 940, carbomer 941, carbomer 1342, polycarbophil, and calcium polycarbophil.

3. A method according to claim 1, wherein the carbomer is carbomer 934P.

4. A method according to claim 1, wherein the organic solvent is selected from benzene, phenol(s), toluene, ethyl acetate, methanol, ethanol, isopropanol, hexane, acetone, chloroform, 1,4-dioxane, dimethyl sulfoxide, methylene chloride, trichloroethylene, 1,2-dichloroethane, carbon tetrachloride, and 1,1-dichloroethene.

5. A method according to claim 1, wherein the organic solvent is benzene.

6. A method according to claim 1, wherein the gaseous fluid has a critical temperature less than about 200° C. and a critical pressure of less than about 10,000 psi.

7. A method according to claim 1, wherein the gaseous fluid is selected from carbon dioxide, sulfur hexafluoride, nitrous oxide, trifluoromethane, tetrafluoromethane, ethane, ethylene, propane, propanol, isopropanol, propylene, butane, butanol, isobutane, isobutene, hexane, cyclohexane, benzene, toluene, o-xylene, ammonia, water, and mixtures thereof.

8. A method according to claim 1, wherein the gaseous fluid is or comprises carbon dioxide.

9. A method according to claim 1, wherein the gaseous fluid further comprises one or more organic solvents, and/or one or more light gases.

10. A method according to claim 1, wherein said method is conducted at a temperature in the range of about 0.8 to about 1.4 times the critical temperature of the gaseous fluid in degrees Kelvin.

11. A method according to claim 1, wherein said method is conducted at a temperature in the range of about 1.0 to about 1.2 times the critical temperature of the gaseous fluid in degrees Kelvin.

12. A method according to claim 1, wherein said method is conducted at a pressure in the range of about 0.5 to about 100 times the critical pressure of the gaseous fluid.

13. A method according to claim 1, wherein said method is conducted at a pressure in the range of about 1 to about 9 times the critical pressure of the gaseous fluid.

14. A method according to claim 1, wherein the gaseous fluid is carbon dioxide and the method is conducted at a temperature of about 31 to 80° C. and at a pressure of about 1,070 to 10,000 psig.

15. A method according to claim 1, wherein the pressure of the gaseous fluid is kept constant during the extraction of the residual organic solvent.

16. A method according to claim 1, wherein the pressure of the gaseous fluid is repeatedly modulated between two or more pressure levels during the extraction of the residual organic solvent.

17. A method according to claim 16, wherein the relative difference between the uppermost and lowermost levels of density of said gaseous fluid at said pressure levels is not more than about 30%.

18. A method according to claim 16, wherein the relative difference between the uppermost and lowermost levels of density of said gaseous fluid at said pressure levels is not more than about 5%.

19. A method according to claim 1, wherein the residual organic solvent present in the carbomer is reduced to a level of less than about 30 ppm.

20. A method according to claim 1, wherein the residual organic solvent present in the carbomer is reduced to a level of less than about 10 ppm.

21. A method according to claim 1, wherein the residual organic solvent present in the carbomer is reduced to a level of less than about 2 ppm.

22. A method according to claim 1, wherein:
the carbomer is carbomer 934P;
the residual organic solvent is benzene;
and the gaseous fluid is carbon dioxide.

23. A method according to claim 22, wherein the pressure of the gaseous fluid is kept constant during the extraction of the residual organic solvent.

24. A method according to claim 22, wherein the pressure of the gaseous fluid is repeatedly modulated between two or more pressure levels during the extraction of the residual organic solvent.

25. A method according to claim 22, wherein the residual organic solvent present in the carbomer is reduced to a level of less than about 30 ppm.

26. A method according to claim 22, wherein the residual organic solvent present in the carbomer is reduced to a level of less than about 10 ppm.

27. A method according to claim 22, wherein the residual organic solvent present in the carbomer is reduced to a level of less than about 2 ppm.

28. A carbomer that has been treated by the method according to claim 1.

29. A carbomer that has been treated by the method according to claim 21.

30. A carbomer that has been treated by the method according to claim 22.

31. A carbomer that has been treated by the method according to claim 27.

32. A suspension comprising a therapeutically active agent and a carbomer according claim 28.

33. A suspension comprising a therapeutically active agent and a carbomer according claim 29.

34. A suspension comprising a therapeutically active agent and a carbomer according claim 30.

35. A suspension comprising a therapeutically active agent and a carbomer according claim 31.

36. A suspension according to claim 32, wherein the therapeutically active agent is selected from meloxicam, ipratropium bromide, tiotropium bromide, oxytropium bromide, albuterol, albuterol sulfate, clenbuterol, fenoterol, beclomethasone diproprionate, insulin, analgesics, anti-cancer agents, antimicrobial agents, antiviral agents, antifungals, antibiotics, nucleotides, amino acids, peptides, proteins, immune suppressants, thrombolytics, anticoagulants, central nervous system stimulants, decongestants, diuretic vasodilators, antipsychotics, neurotransmitters, sedatives, hormones, anesthetics, anti-inflammatories, antioxidants, antihistamines, vitamins and minerals.

37. A suspension according to claim 34, wherein the therapeutically active agent is selected from meloxicam, ipratropium bromide, tiotropium bromide, oxytropium bromide, albuterol, albuterol sulfate, clenbuterol, fenoterol, beclomethasone diproprionate, insulin, analgesics, anti-cancer agents, antimicrobial agents, antiviral agents, antifungals, antibiotics, nucleotides, amino acids, peptides, proteins, immune suppressants, thrombolytics, anticoagulants, central nervous system stimulants, decongestants, diuretic vasodilators, antipsychotics, neurotransmitters, sedatives, hormones, anesthetics, anti-inflammatories, antioxidants, antihistamines, vitamins and minerals.

38. A suspension according to claim 34, wherein the therapeutically active agent is nevirapine.

39. A suspension according to claim 38, wherein the residual organic solvent present in the carbomer is reduced to a level of less than about 2 ppm.
